(19) United States
(12) Patent Application Publication
(10) Pub. No.: US 2008/0015491 A1
(43) Pub. Date: Jan. 17, 2008

(54) RECOVERY CATHETER APPARATUS AND METHOD

(75) Inventors: Nianjiong Bei, Foster City, CA (US); William J. Boyle, Fallbrook, CA (US); Gregory W. Fung, San Mateo, CA (US); Joanna Lubas, Fremont, CA (US); Alexander Nikanorov, Fallbrook, CA (US)

Correspondence Address: FULWIDER PATTON LLP, HOWARD HUGHES CENTER, 6060 CENTER DRIVE, TENTH FLOOR, LOS ANGELES, CA, 90045, US

(73) Assignee: ABBOTT VASCULAR SOLUTIONS, INC., Santa Clara, CA (US)

(21) Appl. No.: 11/828,518

(22) Filed: Jul. 26, 2007

Related U.S. Application Data
(63) Continuation of application No. 11/066,028, filed on Feb. 24, 2005.

(51) Int. Cl. A61M 25/00

(52) U.S. Cl. 604/27

(57) ABSTRACT

A catheter assembly configured for retrieval of medical devices from vasculature. The catheter includes an outer catheter and an inner catheter. The inner and/or outer catheter can include a tapered terminal end portion. A mandrel can be provided to facilitate advancement of the assembly within vasculature.

### BACKGROUND OF THE INVENTION

The present invention relates generally to recovery catheters for use in vasculature. More particularly, the present invention is directed towards recovery catheters for filtering devices and systems which can be used when an interventional procedure is being performed in a stenosed or occluded region of a blood vessel to capture embolic material that may be created and released into the bloodstream during the procedure.

Embolic filtering devices and systems are particularly useful when performing balloon angioplasty, stenting procedures, laser angioplasty or atherectomy in critical vessels, especially in vessels where the release of embolic debris into the bloodstream can occlude the flow of oxygenated blood to the brain or other vital organs, which can cause devastating consequences to the patient. In fact, the embolic protection devices and systems are useful with any vascular interventional procedure in which there is an embolic risk. Recovery catheters are essential to the successful retrieval of such protection systems and thus, to the success of the interventional procedure being performed.

A variety of non-surgical interventional procedures have been developed over the years for opening stenosed or occluded blood vessels in a patient caused by the build up of plaque or other substances on the wall of the blood vessel. Such procedures usually involve the percutaneous introduction of the interventional device into the lumen of the artery, usually through a catheter. In typical carotid PTA procedures, a guiding catheter or sheath is percutaneously introduced into the cardiovascular system of a patient through the femoral artery and advanced through the vasculature until the distal end of the guiding catheter is in the common carotid artery. A guide wire and a dilatation catheter having a balloon on the distal end are introduced through the guiding catheter with the guide wire sliding within the dilatation catheter. The guide wire is first advanced out of the guiding catheter into the patient's carotid vasculature and is directed across the arterial lesion. The dilatation catheter is subsequently advanced over the previously advanced guide wire until the dilatation balloon is properly positioned across the arterial lesion. Once in position across the lesion, the expandable balloon is inflated to a predetermined size with a radiopaque liquid at relatively high pressures to radially compress the atherosclerotic plaque of the lesion against the inside of the artery wall and thereby dilate the lumen of the artery. The balloon is then deflated to a small profile so that the dilatation catheter can be withdrawn from the patient's vasculature and the blood flow resumed through the dilated artery. As should be appreciated by those skilled in the art, while the above-described procedure is typical, it is not the only method used in angioplasty.

Another procedure is laser angioplasty which utilizes a laser to ablate the stenosis by super heating and vaporizing the deposited plaque. Atherectomy is yet another method of treating a stenosed blood vessel in which cutting blades are rotated to shave the deposited plaque from the arterial wall. A vacuum catheter is usually used to capture the shaved plaque or thrombus from the blood stream during this procedure.

In the procedures of the kind referenced above, abrupt reclosure may occur or restenosis of the artery may develop over time, which may require another angioplasty procedure, a surgical bypass operation, or some other method of repairing or strengthening the area. To reduce the likelihood of the occurrence of abrupt reclosure and to strengthen the area, a physician can implant an intravascular prosthesis for maintaining vascular patency, commonly known as a stent, inside the artery across the lesion. The stent is crimped tightly onto the balloon portion of the catheter and transported in its delivery diameter through the patient's vasculature. At the deployment site, the stent is expanded to a larger diameter, often by inflating the balloon portion of the catheter.

Prior art stents typically fall into two general categories of construction. The first type of stent is expandable upon application of a controlled force, as described above, through the inflation of the balloon portion of a dilatation catheter which, upon inflation of the balloon or other expansion means, expands the compressed stent to a larger diameter to be left in place within the artery at the target site. The second type of stent is a self-expanding stent formed from, for example, shape memory metals or super-elastic nickel-titanium (NiTi) alloys, which will automatically expand from a collapsed state when the stent is advanced out of the distal end of the delivery catheter into the body lumen. Such stents manufactured from expandable heat sensitive materials allow for phase transformations of the material to occur, resulting in the expansion and contraction of the stent.

The above non-surgical interventional procedures, when successful, avoid the necessity of major surgical operations. However, there is one common problem which can become associated with all of these non-surgical procedures, namely, the potential release of embolic debris into the bloodstream that can occlude distal vasculature and cause significant health problems to the patient. For example, during deployment of a stent, it is possible that the metal struts of the stent can cut into the stenosis and shear off pieces of plaque which become embolic debris that can travel downstream and lodge somewhere in the patient's vascular system. Pieces of plaque material can sometimes dislodge from the stenosis during a balloon angioplasty procedure and become released into the bloodstream. Additionally, while complete vaporization of plaque is the intended goal during a laser angioplasty procedure, quite often particles are not fully vaporized and thus enter the bloodstream. Likewise, not all of the emboli created during an atherectomy procedure may be drawn into the vacuum catheter and, as a result, enter the bloodstream as well.

When any of the above-described procedures are performed in arteries, the release of emboli into the circulatory system can be extremely dangerous and sometimes fatal to the patient. Debris that is carried by the bloodstream to distal vessels of the brain can for example cause these cerebral vessels to occlude, resulting in a stroke, and in some cases, death. Therefore, although cerebral percutaneous transluminal angioplasty has been performed in the past, the number of procedures performed has been limited due to the justifiable fear of causing an embolic stroke should embolic debris enter the bloodstream and block vital downstream blood passages.

Medical devices have been developed to attempt to deal with the problem created when debris or fragments enter the circulatory system following vessel treatment utilizing any one of the above-identified procedures. One approach which has been attempted is the cutting of any debris into minute sizes which pose little chance of becoming occluded in major vessels within the patient's vasculature. However, it is often difficult to control the size of the fragments which are formed, and the potential risk of vessel occlusion still exists, making such a procedure in the carotid arteries a high-risk proposition.

Other techniques which have been developed to address the problem of removing embolic debris include the use of catheters with a vacuum source which provides temporary suction to remove embolic debris from the bloodstream. However, as mentioned above, there have been complications with such systems since the vacuum catheter may not always remove all of the embolic material from the bloodstream, and a powerful suction could cause problems to the patient's vasculature. Other techniques which have had some success include the placement of a filter or trap downstream from the treatment site to capture embolic debris before it reaches the smaller blood vessels downstream. However, there have been problems associated with filtering systems, particularly during the expansion and collapsing of the filter within the body vessel. If the filtering device does not have a suitable mechanism for closing the filter, there is a possibility that trapped embolic debris can backflow through the inlet opening of the filter and enter the blood-stream as the filtering system is being collapsed and removed from the patient. In such a case, the act of collapsing the filter device may actually squeeze trapped embolic material through the opening of the filter and into the bloodstream.

Certain of the available filters which can be expanded within a blood vessel are attached to the distal end of a guide wire or guide wire-like tubing which allows the filtering device to be placed in the patient's vasculature when the guide wire is manipulated in place. Once the guide wire is in proper position in the vasculature, the embolic filter can be deployed within the vessel to capture embolic debris. The next step then involves removing the captured debris and filter device from vasculature.

Since the efficient and effective retrieval of a filter which has captured vasculature debris can be highly critical to the success of an interventional procedure, the structure of a retrieval catheter must facilitate such retrieval. Accessing a filter or embolic protection device can be a concern where the interventional site is defined by tortuous or narrow anatomy. Interference between the filter and recovery catheter can also occur, where for example, the components become entangled. Identifying an exact location of the recovery catheter with respect to a filter device can also become a critical concern.

Accordingly, what is needed is a recovery catheter that enables structural characteristics specifically designed to facilitate the advancement thereof through narrow and tortuous vasculature to an interventional site. It is also desirable that a superior end portion of the recovery catheter be configured to avoid interferences with a filter device and provides an effective receptacle for the filter.

The present invention satisfies these and other needs.

### SUMMARY OF THE INVENTION

Briefly and in general terms, the present invention is directed towards a recovery catheter for use in vasculature. The recovery catheter can be employed to receive and retrieve various medical devices placed within vasculature of a patient.

In one aspect, the recovery catheter of the present invention is intended to be used to facilitate the retrieval of a filter or embolic protection device. The recovery catheter is thus equipped with a superior end portion configured to accept at least a portion of a filter or embolic protective device.

A system is provided for use in vasculature. The system includes a filter device connected to an elongate member; and a recovery catheter, the recovery catheter including an outer catheter and an inner catheter slideably received in the outer catheter, the outer catheter including a superior end portion sized to receive the filter, and the inner catheter including a lumen that receives the elongate member and a terminal end portion forming a tapered tip. Alternatively, the system can include a filter device and a recovery catheter including an elongate tubular member configured to receive the filter device and a mandrel extending along the tubular member, the mandrel having a variable durometer along its length.

The recovery catheter has an elongate profile and a length sufficient to extend from exterior of a patient to an interventional site within the patient's vasculature. An inferior end portion is designed to be manipulated by a physician or operator during advancement to the interventional site as well as once the site has been accessed.

In one particular aspect, the recovery catheter includes an elongate tubular member having variable flexibility along its length. In one embodiment, the catheter is equipped with a rapid exchange juncture and a highly flexible tip. The device can also be configured with a stopper to facilitate proper positioning of a medical device within the catheter.

In another aspect, the recovery catheter includes an outer catheter and an inner catheter slideably received within the outer catheter. The inner catheter has a tapered terminal end and a guide wire lumen extending substantially its length. The inner catheter can also include a side port that provides access to the guide wire lumen. Likewise, the outer catheter can be provided with a side opening that provides access to the guide wire lumen.

In further aspects, the recovery catheter can include a retractable or removable terminal tip connected to a manipulation wire. The outer catheter can be tapered and the system equipped with a support mandrel having varying stiffness along its length. Further, a diaphragm seal can be placed at an opening that receives a guide wire and the tubing can alternatively be supported by a braided structure. Moreover, a lubricious coating is contemplated to be placed on various components of the system.

In yet another embodiment, the recovery catheter includes a tip having a tapered superior end. The tapered superior end can embody elastic properties so that it has an expanded profile when receiving a filter or embolic protection device.

Other features and advantages of the present invention will become apparent from the following detailed description, taken conjunction with the accompanying drawings, which illustrate, by way of example, the principles of the invention.

### BRIEF DESCRIPTION OF THE DRAWINGS

FIG. 1 is a side view, depicting one embodiment of a recovery catheter of the present invention;

FIG. 2 is an enlarged cross-sectional view, depicting a cross section of the recovery catheter of FIG. 1 taken along lines 2-2;

FIG. 3 is an enlarged cross-sectional view, depicting a cross section of the recovery catheter of FIG. 1 taken along lines 3-3;

FIG. 4 is an enlarged cross-sectional view, depicting a cross section of the recovery catheter of FIG. 1 taken along lines 4-4;

FIG. 5 is a partial cross-sectional view, depicting a catheter assembly including an inner catheter having a tapered superior end;

FIG. 6 is a partial cross-sectional view, depicting the catheter assembly of FIG. 5 with a filter device withdrawn within an outer catheter;

FIG. 7 is a partial cross-sectional view, depicting a catheter assembly with a retractable tip;

FIG. 8 is a partial cross-sectional view, depicting the catheter assembly of FIG. 7 with a filter device withdrawn within an outer catheter;

FIG. 9 is a partial cross-sectional view, depicting an alternative embodiment of a catheter assembly including a tapered mandrel;

FIG. 10a is a perspective view, depicting a catheter assembly including a sealing member;

FIG. 10b is a rotated perspective view, depicting the catheter assembly of FIG. 10a;

FIG. 11 is a partial cross-sectional view, depicting a catheter assembly including a superior end portion having elastic properties; and

FIG. 12 is a partial cross-sectional view, depicting the catheter assembly of FIG. 11 with a filter withdrawn within the superior end portion.

### DETAILED DESCRIPTION OF THE INVENTION

Referring to the drawings, which are provided for example and not by way of limitation, there is shown a recovery catheter for use with a filter or embolic protection device. The recovery catheter of the present invention embodies structural characteristics specifically designed to facilitate advancement through narrow and/or tortuous vasculature. Moreover, the recovery catheter includes a superior end portion configured to provide an effective receptacle for a filter or other medical device and to minimize interference with other components in vasculature.

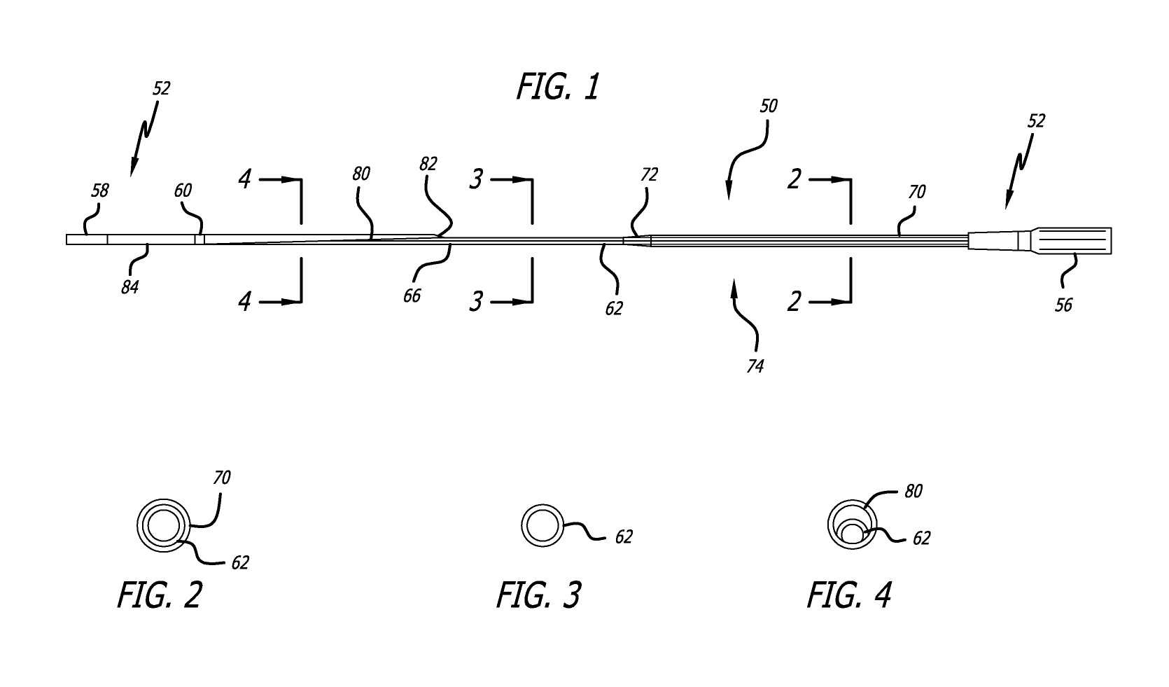

With reference to FIGS. 1-4, there is shown one embodiment of a recovery catheter 50 of the present invention. The recovery catheter 50 is elongate having a length sufficient to extend from outside a patient's body to an interventional site within the patient. The recovery catheter 50 is generally tubular in shape and includes a proximal or inferior end portion 52 and a distal or superior end portion 54. The proximal end portion 52 includes a generally tubular luer or handle 56 which is sized to be threaded over a guidewire or other elongate member of a medical device such as a filter or retrieval basket. The distal end 54 includes a tubular tip 58 made from flexible material.

Extending from luer or handle 56 to a stopper member 60 is a mandrel 62. In one embodiment, the stopper 60 is cylindrical in shape and is positioned along the recovery catheter 50 inferior to the tip 58. The length of the catheter 50 from the tip 58 to the stopper 60 is sized to accept a medical device such as a basket of a medical retrieval device, the stopper 60 acting to limit the extent to which the medical device can be withdrawn within the catheter 50. The mandrel 62 is intended to provide the catheter 50 with the desired flexibility and pushability. In one aspect, the mandrel 62 is contemplated to include tapered sections and to narrow as it extends distally. However, the mandrel 62 can also define a straight tube or a gradual taper either proximally or distally rather than including tapered sections. Moreover, the mandrel can for certain applications, variably increase or decrease in cross-section along its length.

As shown in FIGS. 1-4, the mandrel 62 can include a straight section and a tapered section. The straight section of the mandrel begins at luer or handle 56 and extends to point 66. At point 66, the mandrel begins to taper in a superior direction. Such a configuration can be appreciated by the cross-sectional structure shown in FIGS. 2-4. Moreover, the mandrel 62 is contemplated to be coated with a plastic elastomer. In one particular embodiment, the mandrel 62 is made from stainless steel and is coated with Pebax. The Pebax coating or jacket necks down to fit snugly about the tapered portion of the mandrel 62.

The catheter 50 is also equipped with a hypotube 70 coated with Pebax material. The hypotube 70 extends distally from the luer 56 to a transition point 72 at which the hypotube 70 necks down and terminates. The hypotube 70 and the mandrel 62 cooperate to provide a proximal section 74 of the catheter 50 with desired flexibility and pushability. With reference to FIG. 2, the device can include four layers of material including the coatings. The catheter 50 can also include two layers of material (including coating) as exemplified in the cross-sectional view of FIG. 3. A four layer cross-sectional structure is also found at the area of the stopper 60.

Between transition 72 and point 66, the coated mandrel 62 alone provides the desired flexibility and pushability. At point 66, the mandrel 62 begins to narrow and is surrounded by an outer tube or catheter 80. At point 66, a lateral space or opening 82 is provided between the mandrel 62 and outer tube 80. This opening 82 is designed to operate as a rapid exchange junction through which a wire or similar structure of a medical device can be threaded.

The outer tube 80 extends distally beyond the stopper 60 and is joined to the flexible tip 58. The distal most portion 84 of the outer tube 80 along with tip 58 define a cavity for receiving the medical device.

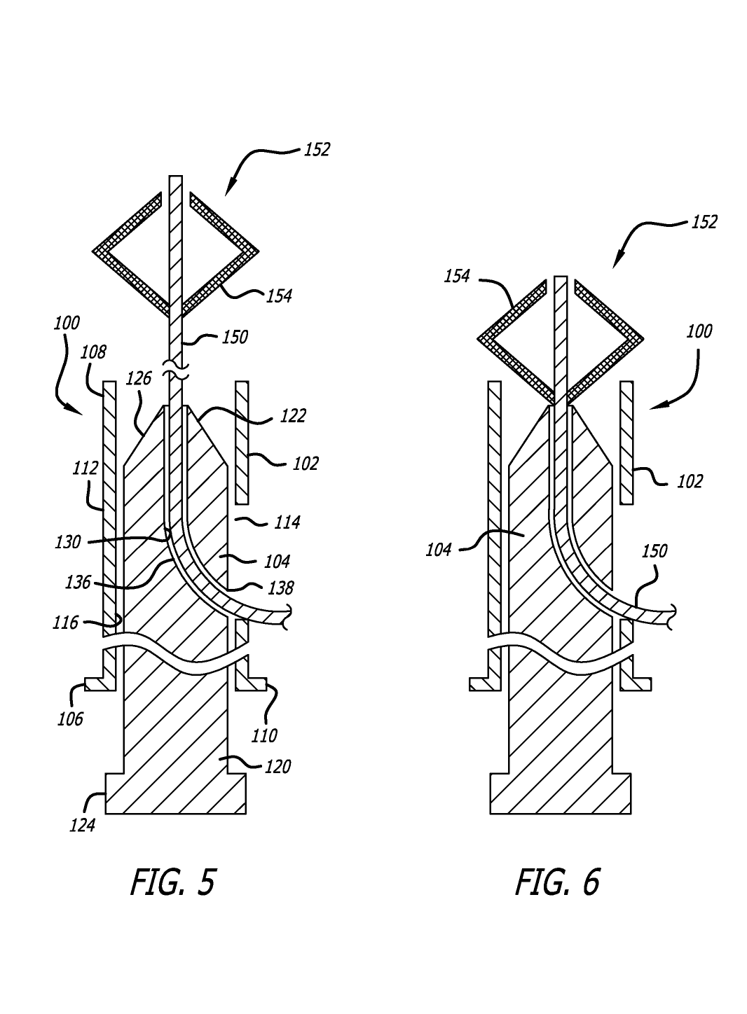

Referring now to FIGS. 5 and 6, in another embodiment, a recovery catheter 100 of the present invention includes an elongate outer catheter 102 and an elongate inner catheter 104. The outer catheter 102 has a generally tubular configuration and includes an inferior or proximal end portion 106 and a superior or distal portion 108.

The proximal end portion 106 of the recovery catheter 100 further includes a handle or luer assembly 110 configured specifically for grasping and manipulation by an operator. Along a midsection 112 of the recovery catheter 100, a rapid exchange sideport 114 can be formed. A lumen 116 extends the length of the recovery catheter 100 from its proximal end portion 106 to the distal end portion 108. The lumen 116 is contemplated to be in communication with the sideport 114. In an alternative embodiment, the recovery catheter 100 can lack sideport 114 where a rapid exchange approach is not contemplated.

The inner catheter 104 includes an inferior or proximal end portion 120 and a superior or distal end portion 122. The proximal end portion can additionally be equipped with a luer or handle assembly 124 for manipulation by an operator. Moreover, the handle assembly 124 can be configured to include locking structure that cooperates with handle assembly 110 of the outer catheter. Also, the distal end portion 122 of the inner member 104 is contemplated to have a tapered or narrowing profile 126. Such tapering can take on various forms including a generally conical profile or can assume other asymmetric shapes. Moreover, the tapered profile 126 of the inner catheter provides a surface for advancing the inner catheter 104 and outer catheter 102 through vasculature. That is, the tapered leading end 126 aids in the negotiation of tortuous and difficult anatomy.

A lumen 130 is contemplated to extend along a portion of the inner catheter 104. In one aspect, the lumen 130 can extend the full length of the inner catheter 104 from its proximal end portion 124 to its distal end portion 122. However, as is shown in FIGS. 5 and 6, the lumen 130 can alternatively extend from the distal end portion 122 of the inner catheter 104 to a transition junction 136 where the lumen curves and exits a sidewall of the inner catheter 104. The exit point 138 can be placed along a midsection of the inner catheter and is arranged to be in alignment with the sideport 114 of the outer catheter 102 to thereby provide a rapid exchange conduit.

The inner catheter lumen 130 is designed to receive a guide wire or other elongate structure 150 of a filter or embolic protection assembly 152. The filter assembly 152 includes the wire 150 as well as a basket assembly or receptacle 154 attached to the wire 150 at a superior or distal end of the wire 150.

The elongate member or wire 150 of the filter device 152 when received within the recovery catheter 100 is contemplated to extend in an inferior direction to the operator. Manipulation of the elongate member 150 accomplishes the relative longitudinal movement between the filter assembly 152 and the inner 104 and outer 102 catheters. Such action enables the capture and retrieval of emboli or other material found within vasculature.

In one particular embodiment, the distal end portion 122 of the inner catheter 104 is configured to accomplish centering the wire 150 and filter assembly 152 itself within the outer catheter 102. In this way, the filter assembly 152 can be effectively withdrawn within the outer catheter 102 and the outer catheter facilitates the uniform or other approach to collapsing the basket 154 within the outer catheter or other desirable engagement between the outer catheter and the basket 154.

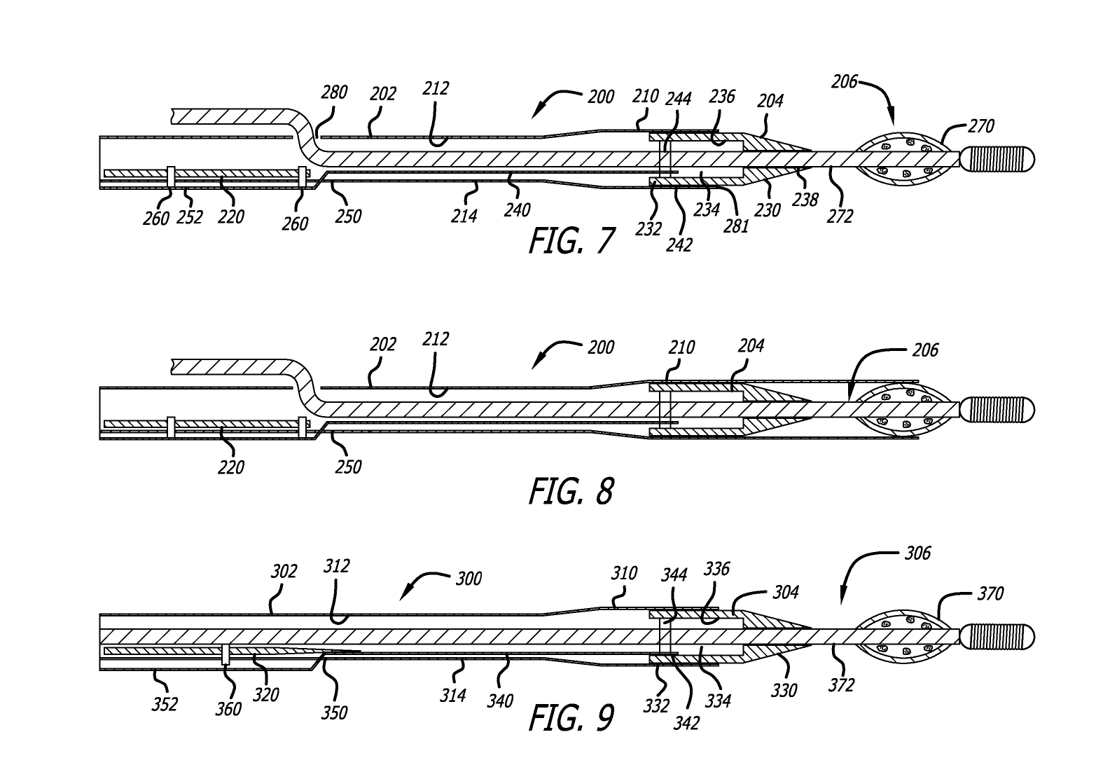

Turning now to FIGS. 7 and 8, further aspects of a recovery catheter 200 of the present invention are depicted. In this embodiment, the recovery catheter 200 includes an elongate outer catheter 202 and a retractable tip assembly 204. The recovery catheter 200 is sized and shaped to receive a filter or embolic protection assembly 206.

The outer catheter 202 has a generally tubular configuration and includes an inferior or proximal end portion (not shown) and a superior or distal end portion 210. The inferior end can be equipped with conventional luers or handles to facilitate manipulation of the recovery catheter 200. Extending the length of the outer catheter 202 is a lumen 212 sized to receive both of the retractable tip assembly 201 and the filter assembly 206.

In one aspect of the invention, the outer catheter 202 has a tapered profile. The distal end portion 210 has a larger profile than the midsection 214 or inferior portion of the catheter 200. The larger profile portion provides a space for receiving the filter assembly 206 and tapers down to the profile defined by the midsection portion 214.

The recovery catheter 200 is also provided with a mandrel 220. The mandrel 220 provides the recovery catheter 200 with desired axial flexibility characteristics as well as desirable column strength which enhances the pushability of the recovery catheter through vasculature. The mandrel 220 can extend any predetermined length of the recovery catheter 200 and can be configured to be affixed to the recovery catheter or to move longitudinally with respect thereto. Having such flexibility in design, the recovery catheter 200 can have variable durometer during various stages of use.

The retractable tip 204 includes a superior or distal end portion 230 and an inferior or proximal portion 232. An internal bore 234 extends the length of the retractable tip 204 and includes a proximal portion 236 having a larger diameter or cross-sectional profile and a distal portion 238 having a smaller diameter or cross-sectional profile. The superior end portion 230 of the retractable tip 204 is tapered or narrowed in a uniform or variable manner to provide the recovery catheter 200 with a desirable leading profile.

A manipulation wire 240 is attached at a superior end 242 to the retractable tip 204 via a ring 244 or other connecting structure. The manipulation wire 240 extends in an inferior direction to an operator. The manipulation wire 240 can extend within the lumen 212 of the outer catheter 202 or can extend through a rapid exchange sideport 250 formed in the outer catheter 202. The sideport 250 can be a simple hole formed in the wall of the outer catheter 202 or can be formed by overlapping concentrically arranged end portions of a pair of tubes leaving a space for the egress of the manipulation wire 240.

Where the manipulation wire 240 exits a rapid exchange port 250, a proximal or inferior portion 252 extends along side an exterior of the outer tube. One or more rings 260 can be provided to guide the manipulation wire 240 along the exterior of the outer catheter 202. The guiding rings 260 can be attached to the recovery catheter 200 itself or can be affixed to the mandrel 220.

The filter assembly 206 includes a filter body 270 attached to a superior end portion of a guide wire 272. The guide wire 272 extends in an inferior direction through both the retractable tip 204 and the outer catheter 202 when the device is assembled for use. Although an over-the-wire approach is also contemplated, the recovery catheter can be provided with a rapid exchange junction 280 formed in a sidewall of the outer catheter 202. The rapid exchange junction 280 is contemplated to be spaced circumferentially separate from the rapid exchange sideport 250 and can be formed in a similar manner. It is also contemplated, however, that the guide wire 272 of the filter assembly 206 can share the same rapid exchange port as the manipulation wire 240.

The filter guide wire 272 is further configured to pass through the retractable tip in a manner which facilitates centering the filter body 270 within the outer catheter. This can be accomplished by centering the bore 238 within the superior portion 230 of the retractable tip 204. Such an arrangement aids in uniformly collapsing the filter body 270 within the outer catheter 202. The terminal end 281 of the outer catheter can be perpendicular to a longitudinal axis of the outer catheter 202 or can alternatively be angled with respect thereto. Such terminal ends are adapted to facilitate collapsing the filter body 270 in a desirable manner. Various filter body 270 designs can be received or captured by the recovery catheters of the present invention.

Turning now to FIG. 9, there is shown a catheter 300 which includes a number of structural details in common with the recovery catheter shown in FIGS. 7 and 8. The recovery catheter 300 shown further includes a tapered mandrel 320 rather than a mandrel having an uniform profile.

In operation, the filter assembly 270, 370 is placed in vasculature adjacent an interventional site. A recovery catheter 200, 300 is advanced over the filter assembly 270, 370 or it can be delivered within vasculature contemporaneously with the filter assembly. The tapered retractable tip 204, 304 of the recovery catheter 200, 300 facilitates the advancement within and placement of the assembly at the interventional site. Subsequent to manipulating the filter assembly to capture material found in vasculature, the filter guide wire 272, 372 and the recovery catheter manipulation wire 240, 340 are pulled proximally to withdraw the retractable tip 204, 304 and the filter body 270, 370 within the recovery catheter 200, 300. The increased profile of the superior end portion 210, 310 of the recovery catheter 200, 300 provides space for effectively recovering the filter containing embolic debris or other material collected from vasculature.

In certain circumstances, it may be necessary to elicit the help of an insertion tool to insert the catheter of the present invention into vasculature. For example, when threading the recovery catheter 200, 300 over a guidewire of a filter device, and into an introducer device already placed within vasculature for the purpose of providing access thereto, a tubular funnel (not shown) can be employed to aid in advancing a superior or distal end of the catheter into an inferior or proximal end of the introducer device. The funnel would include a larger end and a smaller end, the smaller end sized to fit within the introducer device. The larger end is designed to receive the distal end of the recovery catheter and to facilitate the advancement thereof into the introducer device. The funnel can include a longitudinal slit to allow the placement of the device on a guidewire and can gradually flare or increase in diameter in a stepped fashion from the small end to the larger end. In use, the funnel is first threaded or placed over the guidewire of a filter device followed by the threading of the recovery catheter over the guidewire and through the funnel and into the introducer. As the recovery catheter is used to capture a basket or receptacle of the filter or other medical device, the funnel is withdrawn from engagement with the introducer device. Upon withdrawal of the recovery catheter and filter, the funnel can be advanced to engagement with the introducer and utilized again to aid in the egress of the filter or medical device from the introducer device.

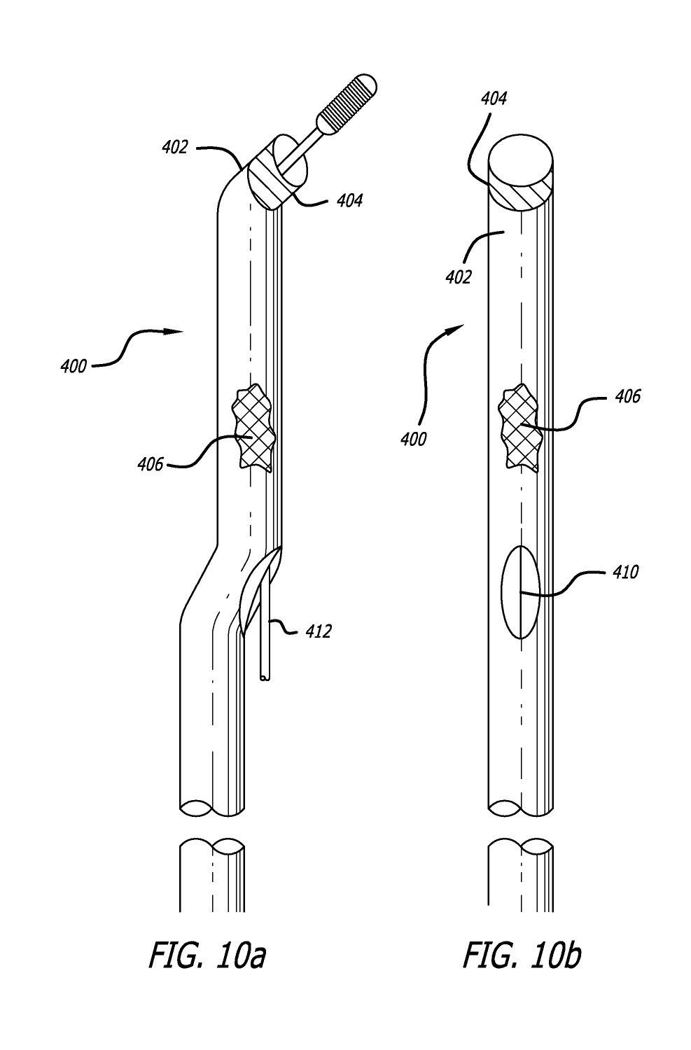

With reference to FIGS. 10a and 10b, there is shown another embodiment of a recovery catheter 400 of the present invention. The recovery catheter 400 is tubular and elongate in configuration. A proximal or inferior end (not shown) can be configured with various conventional structures for manipulating the device. A superior or distal end portion 402 is configured with a radiopaque marker band 404. The marker band may consist of a biocompatible polymer loaded with a radiopaque metallic oxide such as bismuth oxide or similar biocompatible radiopaque oxide. Alternatively, a pair of radiopaque markers can be attached by gluing, melting or swaging to the recovery catheter 400. The longitudinal distance between the two markers can be set to equal a length of a medical device which is desirable to be withdrawn within the recovery catheter 400 to thereby assure that complete recovery is achieved. Thus, the pair of markers will coincide with or extend beyond markers placed on the device being withdrawn into the recovery catheter 400. The recovery catheter can also be provided with a braided substructure 406 to enhance column strength for pushability or to provide a desired axial flexibility and torquability. The braided substructure 406 can be sandwiched between layers of catheter material or can be adhered to an inner wall of the catheter 400.

The recovery catheter 400 is further equipped with a sealing diaphragm 410. The diaphragm 410 can be incorporated into any of the recovery catheters of the present invention for a number of purposes. For example, the diaphragm 410 can form the path for the rapid exchange of a guide wire 412 alone or one which is equipped with a medical device such as an embolic protection device or filter. Such an arrangement will aid in permitting an operator to perform contrast injections for the positioning of the device during an interventional procedure or for conducting an aspiration of the catheter.

Additionally, the guide wire 412 can be coated with a lubricous substance to reduce friction between the guide wire 412 and the recovery catheter 400. The lubricous coating can be PTFE or similar flouroethylene coatings, paralene or other hydrophilic coatings. The design goal being to facilitate the smooth tracking of the recovery catheter over the guide wire. The overall length of the catheter can be on the order of 100-140 cm with the rapid exchange working portion having a length of up to 10 cm to 30 cm or more.

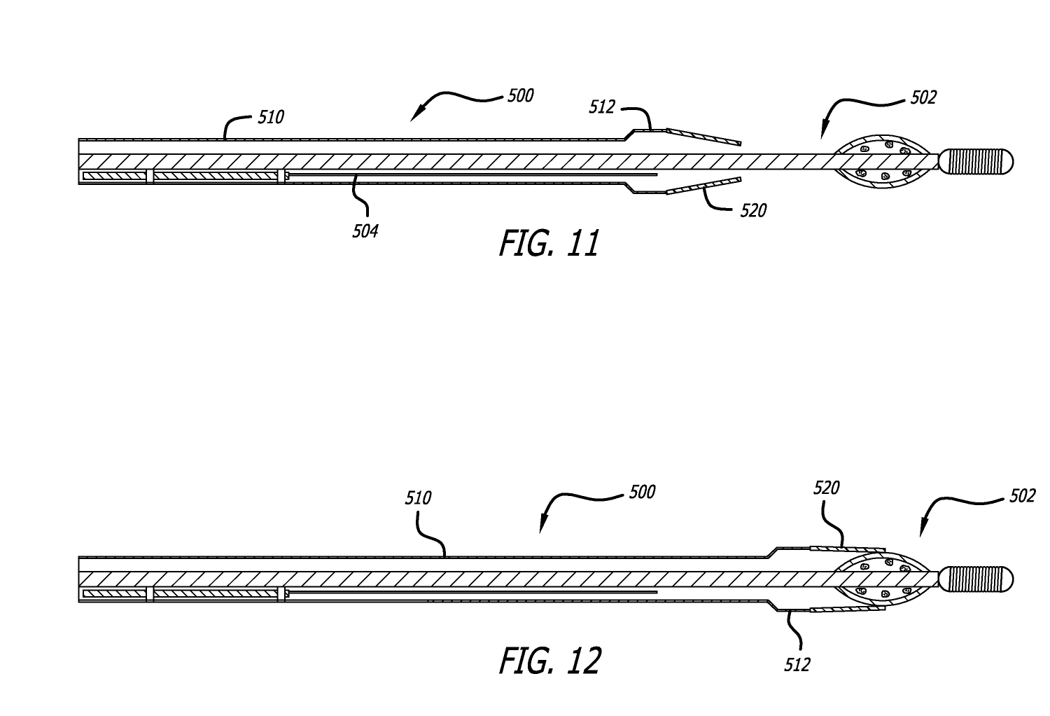

In yet another aspect of the invention (see FIGS. 11 and 12), there is provided a recovery catheter 500 for use in retrieving an embolic protection or filter device 502. As with earlier described aspects of the invention, the recovery catheter 500 can be equipped with a mandrel 504 having variable durometer along its length. The mandrel can be affixed to an outer catheter 500 or can be independently manipulatable to provide further flexibility and column strength and axial flexibility. This mandrel can also be covered with plastic elastomers.

Additionally, the outer catheter is provided with a conventional proximal or inferior end portion (not shown) and a distal or superior end portion 512. The outer catheter can be tapered to thereby provide a distal end portion with a larger profile than other portions of the outer catheter. Again, the larger profile of the distal end portion 512 provides a sufficient space to retrieve a filter device 502 containing collected material. Moreover, as previously described, the recovery catheter 500 can be configured to assume an over-the-wire arrangement or one that takes advantage of aspects of a rapid exchange arrangement.

The distal end region 512 of the recovery catheter can further include a terminal end portion 520 having elastomeric characteristics. That is, the terminal end portion can be made from material which can expand to accept a filter or other medical device 502 yet can assume a smaller profile when unconstrained. In this way, a more desirable tapered profile of the terminal end portion 520 can be used to aid the advancement within vasculature and then facilitate securely receiving a filter device upon withdrawal of the recovery catheter 500 from vasculature.

In one particular embodiment, the outer catheter 510 of the recovery catheter can include a proximal portion made from Pebax 63D-Pebax 72D. The tapered tip 520 can be made from Pebax 25D material. By gradually or abruptly transitioning from Pebax 72D to Pebax 63D along the length of the device, the reduction of material modulus can therefore play a significant role in the advancement through vasculature as well as in achieving high kink resistance. The preferred inner diameter of the distal end tip is about 0.038-0.045 inches and has a wall thickness greater than or equal to 0.003 inches. The length of the distal tip should be less than approximately 18 mm. Additionally, the mandrel is contemplated to be covered with a necked Pebax 72D extrusion and is contemplated to aid in a smooth transition of device durometer.

Accordingly, the present invention is directed towards a recovery catheter embodying structural characteristics specifically designed to facilitate advancement through narrow and tortuous vasculature as well as to effectively receive and remove medical devices from within vasculature. In specific embodiments, the present invention is employed to recover filters or other embolic protection devices containing captured material from vasculature. However, the present invention can be used in conjunction with any medical device and furthermore, the various disclosed details and aspects of the present invention can be applied to each of the contemplated embodiments to create a device having characteristics which are desirable for a particular application.

Thus, it will be apparent from the foregoing that, while particular forms of the invention have been illustrated and described, various modifications can be made without parting from the spirit and scope of the invention.

1-21. (canceled)

22. A system for use in vasculature, comprising:
a filter device connected to an elongate member; and
a recovery catheter, the recovery catheter including an elongate tubular member configured to receive the filter device and a mandrel extending along the tubular member, the mandrel having a variable durometer along its length.

23. A system for use in vasculature, comprising:
a filter device connected to an elongate member; and
a recovery catheter, the recovery catheter including a first section having four layers of material and a second section with two layers of material.
